(19) United States
(12) Patent Application Publication
(10) Pub. No.: US 2020/0365281 A1
(43) Pub. Date: Nov. 19, 2020

(54) VIDEO CONFERENCING AND REAL-TIME SESSION BRIDGING FOR SERVICE DELIVERY USING DRONE TECHNOLOGY

(71) Applicant: Adib Haque, Sydney (AU)

(72) Inventor: Adib Haque, Sydney (AU)

(21) Appl. No.: 16/909,840

(22) Filed: Jun. 23, 2020

Related U.S. Application Data
(63) Continuation-in-part of application No. 15/716,448, filed on Sep. 26, 2017.

(30) Foreign Application Priority Data
May 8, 2020 (SG) 10202004249Q

(51) Int. Cl. G16H 80/00; H04N 7/14; H04N 7/15; G16H 40/67

(52) U.S. Cl. CPC G16H 80/00; G16H 40/67; H04N 7/155; H04N 7/142

(57) ABSTRACT

A system for video conferencing and real-time session bridging for services delivery is provided. The system comprises a processor, a memory, and an application that dispatches, based on receiving a request for assistance at a remote location, an unmanned aerial vehicle to the remote location, the vehicle containing two-way communication functionality. The system also joins a communication session initiated by the vehicle. Based on information exchanged during the communication session, the system also determines a provider device to assist a user of the vehicle causes the provider device to be joined to the communication session. The unmanned aerial vehicle is a drone device programmed to depart the remote location when the communication session ends. A user of the provider device renders assistance via at least one of voice, video, and text media to the user of the vehicle during the communication session.

### FIELD

The present disclosure is in the field of service delivery via wireless communications. More particularly, the present disclosure provides systems and methods of delivering service to remotely located parties using drones and wireless technology.

### BACKGROUND

Persons in need of care and assistance may not have access to communications facilities due to being remotely located or unable to access telephones, computers or pharmacies. If such persons reside in rural or isolated areas, they may not have access to healthcare and other services routinely enjoyed by urban and suburban persons. In the context of a pandemic where the population is in isolation, such services might be required even in an urban setting. They may also may not easily be able to take delivery of medications and other important items.

Transportation in rural or underprivileged areas may be slow and unreliable with poor road and railway infrastructure inhibiting movement and delivery of needed medications, for example. Service providers such as physicians, attorneys, and financial advisors may not wish to locate their practices in such areas.

In emergencies, for example during or after a natural disaster or civil unrest, a person may have lost voice and data connectivity to the outside world. Such persons may need to establish contact with authorities and request assistance including delivery of medical care. During a pandemic, persons may be quarantined and unable to travel and need to remotely receive care.

A remote patient may need to maintain periodic contact with a healthcare provider and confirm compliance with a treatment regimen, and/or report their health status (symptoms, blood pressure readings, etc.) to the healthcare provider. The person may not have access to secure communications capabilities and may therefore be unable to make such required contact.

### SUMMARY

Systems and methods described herein provide for a remotely located person using a received unmanned aerial vehicle or drone device to initiate a video-over-IP or other communication session with a triage service center to request assistance, such as healthcare assistance. The service center provides a triage operator using a web console to communicate with the calling patient and diagnose the patient's condition. From an extensive listing of professional healthcare providers, the triage operator selects an available provider who is qualified in the specialty needed to assist the calling patient.

The triage operator, with the patient in a brief hold state, contacts the qualified and available provider and joins the provider to the call. The patient is then connected with the provider and the provider may then begin a direct diagnosis and development of treatment actions. The triage operator may remain on the line or may drop off.

The drone device sent to the remote party may carry a testing kit and medication that can be dispensed to the patient or other remote person. The drone device may record the person self-testing, which may be required for offenders. The sample is carried back by the drone to the service center or lab. The drone device has an integrated LCD and microphone to talk to doctors remotely or triage. An infrared thermometer may be integrated, and other diagnostics may be sent back in real time to a provider device.

During a lockdown, self-isolation, or quarantine situation when a pandemic may be raging through a population, systems and methods provided herein may allow an isolated and sheltered person to receive medication and transmit test results.

An application programming interface (API) executing on backend servers associated with the service center handles incoming video and other communications from drone devices in use by patients, provider devices, web consoles and others. The API tracks events (call sessions) in real time and updates the spectrum of devices and instances of the web console. The API may assist in accessing databases for information about ailments and treatments and distributing the information. A customized architecture using Session Traversal Utilities for NAT (STUN), Traversal Using Relays around NAT (TURN), and Interactive Connectivity Establishment (ICE) is also provided herein to handle problems associated with Network Address Translation (NAT).

### BRIEF DESCRIPTION OF THE DRAWINGS

FIG. 1 is a block diagram of a system of video conferencing and real-time session bridging for service delivery using drone technology according to an embodiment of the present disclosure.

FIG. 2 is a diagram of a system and method of video conferencing and real-time session bridging for service delivery using drone technology according to an embodiment of the present disclosure.

FIG. 3 is a diagram of a system and method of video conferencing and real-time session bridging for service delivery using drone technology according to an embodiment of the present disclosure.

FIG. 4 is a diagram of a system and method of video conferencing and real-time session bridging for service delivery using drone technology according to an embodiment of the present disclosure.

FIG. 5 is a bottom perspective view of an example drone device for use in a system and method of video conferencing and real-time session bridging for service delivery using drone technology according to an embodiment of the present disclosure.

FIG. 6 is a front perspective view of the drone device of FIG. 5.

FIG. 7 is a rear perspective view of the drone device of FIG. 5.

### DETAILED DESCRIPTION

Systems and methods described herein provide for an unmanned aerial vehicle or drone device to be dispatched to a remote, isolated, or stranded party in need of assistance. The drone device is equipped with two-way communications functionality. The remote party receiving the drone device uses the device to initiate a call for assistance to a triage service center and then be connected with a specialist. The remote party may initially describe his/her illness or problem to a triage operator.

With the remote party still on the line and the call active, the triage operator uses a web console to access a plurality of tools to search databases of providers of the type of services needed by the remote party. When the triage operator locates a provider with the specialized skills to address the problem reported by the remote party, the triage operator may contact the provider and cause the provider to be joined to the ongoing call. The provider and the remote party may then communicate directly.

In an embodiment, the remote party may be a sick or injured person without telephone or computer service. A first responder or family member may send an SMS or text message to the triage service center alerting the center of the remote party's location and need for specialist care. The triage operator then dispatches the drone device to the location of the remote party. The remote party uses the two-way communication functionality of the drone to initiate a call to the triage center. As noted, the triage operator, after selecting a qualified provider for example a specialist in the area of the remote party's ailment, contacts the selected provider and joins the provider to the call. The drone allows the remote party to receive care from the provider.

The remotely located party, having received the drone device, causes the drone device to initiate a VoIP or other communications with the triage service center. The triage operator uses a web console to access various diagnostic and care services. Healthcare providers with various specialties coincidentally advertise their availability that are updated in a dynamic fashion and visible to triage operators on their web consoles.

An architecture is provided that promotes video forwarding via soft-switch and video-over-IP for such patient mobile devices that may be situated in lower bandwidth (3G/4G) areas. The triaging function may be a call center staffed by professionals able to understand and converse with the caller. The triaging function may alternatively be entirely automated and be able, through voice and facial recognition as well as analysis of other input, to identify calling patients' needs to select care providers to assist the calling patients.

The triage operator, with the needy person waiting, accesses a software web console connected to a backend server function and numerous databases to draw upon provider information. In the case of healthcare delivery, the databases contain information about many ailments, their symptoms, and their treatments including the types of providers that may be best suited to treat the remote party's condition.

With the communications session initiated by the remote party running and the party waiting, the triage operator examines listings of providers who are designated as currently available and exhibiting specializations in the party's area of need. Similar to a digital video switchboard operator, the triage operator then performs bridging of the initial call by the drone device to the triage operator with the device used by the healthcare provider. The triage operator effectively conferences the provider device into the call initiated by the drone device.

At least one application programming interface (API) is provided on backend servers that the triage operator and the provider devices used by healthcare providers regularly access for the services provided herein. The parties subscribe to the APIs for events associated with systems and methods provided herein and can send actions, i.e. requests, to the APIs. Access to the APIs permits the web console of the triage operator to receive incoming requests from drone devices, search databases, locate and select available and qualified healthcare providers, and cause drone devices of calling patients to be connected to healthcare providers selected to provide assistance as described herein.

When the patient or other person in need is finished with the drone device, the drone device can be activated to return to the triage server or other location. Such activation can be invoked on site by the patient or remotely by the triage operator.

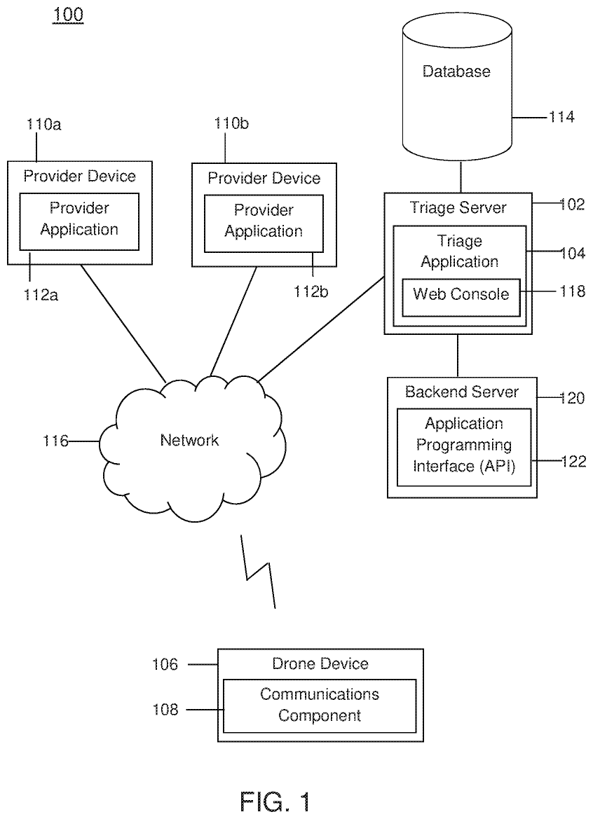

Turning to the figures, FIG. 1 is a block diagram of a system of video conferencing and real-time video session bridging for healthcare delivery in accordance with an embodiment of the present disclosure. FIG. 1 depicts the basic components and interactions of a system 100 in a simplified logical as opposed to physical structure. System 100 comprises a triage server 102, a triage application 104, a drone device 106, a communications component 108, provider devices 110a, 110b, provider applications 112a, 112b, a database 114, and a network 116. System 100 also comprises a web console 118, a backend server 120, and an application programming interface (API) 122.

The triage server 102 and the triage application 104 may be referred to as the server 102 and the application 104, respectively, for brevity purposes. Remarks about any of provider devices 110a, 110b apply to all of provider devices 110a, 110b, respectively, unless otherwise noted

The functionality of the server 102 may in some embodiments reside and execute on a plurality of such computers which may be situated in a plurality of geographic locations. Similarly, in embodiments a plurality of instances of the application 104 may be in effect and may execute on more than one server 102 across more than one geographic location.

A remotely located person may not have telephone or computer communications for various reasons and need assistance. In an emergency, a responder, family member, or other concerned person may contact the triage application 102 on behalf of the person and request assistance. The triage application 102 may then send the drone device 106 to the location of the remotely located person. The location may not be a physical street address but may be global coordinates or some other way of describing the location such that the drone device 106 can reach the person needing assistance.

The drone device 106 is programmable to travel to the location of the remotely located person. The communications component 108 of the drone device 106 allows the person to call the triage server 102 and speak to a triage operator. A triage operator, in some embodiments a human, answers the VoIP or other call from the drone device 106, interviews the calling person, and may open a record using the web console 118. The triage operator, using the web console 118, searches the database 114 for providers who may be able to assist the person. If the person's problem is related to health, the providers may be physicians or other healthcare professionals.

The communications component 108 executing on the drone device 106 allows for VoIP calling as well as video calling. The communications component 108 also allows for still images to be taken and transmitted in both directions as well as transmission of previously recorded video content and text material.

Healthcare providers, for example physicians of various specialties, may use provider devices 110 executing the provider application 112 to announce their availability to handle calls referred by the server 102. The triage operator, with the patient still waiting, uses the web console 118 to consult a dynamically updated portion of the application 104 to determine availabilities of specialists and chooses the specialist best suited to assist the calling patient. The triage operator may consult the database 114 and may access various search engines and electronic tools in performing diagnoses.

The triage operator establishes contact with the chosen provider and conferences the provider and the patient calling via the drone device 106. The session of the calling patient is extended such that the provider using the provider device 110 is brought into the session. The triage operator may drop out of the call once the patient and the provider are joined or alternatively may stay on the call to monitor and update a record of the incident or case.

The session internet protocol (SIP) may be an element of the interactions between components provided herein. Other protocols such as WebRTC may also be used. System 100 also comprises devices not explicitly depicted in FIG. 1 that may use STUN and TURN. Session Traversal Utilities for NAT (STUN) is a standardized set of methods, including a network protocol, for traversal of network address translator (NAT) gateways in applications of real-time voice, video, messaging, and other interactive communications. Traversal Using Relays around NAT (TURN) is a protocol that assists in traversal of network address translators (NAT) or firewalls for multimedia applications.

Technical and business issues facing mobile service providers and others include the implementation by mobile network operators of asymmetric NAT to prevent voice-over-internet protocol (VOIP) operators consuming a lot of bandwidth. Systems and methods provided herein may assist in circumventing the network operators' actions by using the STUN/TURN/ICE architecture and relay servers to result in a successful video call. The provided architecture of SIP/NAT/TURN allows for 4G to 4G video using open source technologies as opposed to proprietary signaling used by such applications as Skype and Facetime.

Systems and methods further allow for calls originated by the drone device 106 to be captured/transferred at the triage stage and analyzed in real time. Previous implementations available from such parties such as Cisco/Polycom may not allow for in-built video switching such as provided herein.

The present disclosure effectively provides an expert/matching system via video which might not have been possible without the triaging step at the web console 118 (whether manual with a human triage operator, automated, or a combination of these). Further, once video provided by at least the drone device 106 is captured using, for example AWS, analytics and deep learning integration, for example adding transcription modules, may be applied to realize further value from the data.

The drone device 106 connecting with the triage server 102 may be logged in to a SIP server which may be a component of the server 102. When the remote person calls the triaging server 102, the SIP server will notify a triage operator at the web console 118 with an operation that is called an invite. The invite contains client ID (SIP ID) medium to communicate with drone device 106, i.e. Host IP, Server Reflexive IP found from a STUN server and relay server IP (TURN) with port numbers. The STUN server provides client NAT information such as host IP, server reflexive IP and port number.

A Keep-Alive header may be used to maintain connections. Network Address Translation (NAT) handles signaling phases of at least one SIP server.

Regarding TURN, when P2P connection fails with Host and Server reflexive IP (using Interactive Connectivity Establishment (ICE)) then TURN server may be used for calling. Audio/video data typically goes through TURN server. A dynamic algorithm that is included in the application 104 is used to detect the network connectivity and provide the optimal video resolution delivery. As systems and methods provided herein may be 4G, most operators block P2P connections. When the remote person's call effectively is forwarded to provider device 110 by the API 122 initiating a second voice-over-IP session on behalf of the drone device 106 to the provider device 110, a SIP server component of the application 104 contacts an address of the provider device 110. Upon connection of the provider device 110 with the drone device 106 which has effectively been on hold, the drone device 106 may be disconnected from the triage server 102 and the remote person and healthcare provider may then begin conversing.

The present disclosure provides systems and methods that may be similar to a digital video switchboard in matching and bridging video sessions to the provider device 110. A web server that may be a component of the application 104 tracks and saves records of the remote person, the provider, and their interactions to at least one Amazon Web Services (AWS) location that may be the database 114.

Real-time tracking of video calls may take place in an AWS cloud or other storage environment. Case notes and case history may also be stored. The server 102 or other device may handle, where applicable, payment from remote callers or others with payment comprising mobile cash or TopUp. Mobile cash and TopUp may be more prevalent than the use of credit cards in emerging markets. The triage operator would validate that a TopUp transfer has been received to verify payment for the session.

Previous implementations that use applications such as Skype and Facetime may provide fixed quality/resolution. Systems and methods provided herein provide for setting appropriate video quality dynamically based on speed of the network involved.

Machine learning and deep learning instances on AWS may splice captured video content and apply NLP (Natural Language Processing) tools and practices to data extracted from the video content. This material may be used to supplement case notes associated with the subject patient.

A pathology module may be a component of the drone device 106. Such a pathology module may allow for data such as blood tests and x-rays to be uploaded and linked/attached to the patient's records for viewing by the care provider's or by an authorized third party.

The triaging function of triage operators working with the application 104 may permit vetting and authorizing of the session that the patient wishes to have with a care provider. Unlike typical instant messaging in which users may add, message, and call each other when they choose, the triaging function provided herein is a virtual control for authorized sessions only. The provider device 110 can also end the video session or transfer the video call back to the triaging function for administrative follow up and documenting of next steps.

While discussion herein has been directed primarily to healthcare applications, systems and methods provided herein may be used in non-healthcare applications. A party in need of legal services might be placed in touch with an attorney or legal aid organization for community assistance centers. A student in need of assistance may contact tutors via the server 102 and application 104. A person or family in need of counseling may contact a qualified counselor for assistance.

In embodiments, systems and methods provide for confidentiality and protection of privacy of all parties involved. Local, state/provincial and federal/national laws receive full compliance. Controls may be built into the application 104, the patient module 108, and the provider application 112 to provide such protections, privacy and regulatory compliance.

At a logic level the code of the application 104 may comprise at least triage server 102 and components of video server functionality. The triage server 102 and the API 122 perform call forwarding and transferring. Video server functionality provided by the triage server 102 may comprise at least one algorithm that determines an appropriate video codec/resolution that may depend in part on the location of the drone device 106. The video server functionality may also detect the quality of the bandwidth of the present session and based at least in part thereon then establish the appropriate video connection.

After triaging is complete, based on global positioning system (GPS), the triage application 104 may alert care providers of the specialty needed within a specific geographic radius to handle the patient's call. In some cases, certified telemedics with a radius can visit the patient with their equipment and gather readings such as blood pressure, glucometer, or electrocardiogram. These telemedics may be vetted in a similar fashion as are the doctors. If there is a concern in the readings. which are collected on a frequent basis (additional service) then an alert goes out to the doctor and/or triage server 102 which follows up with the patient. A healthcare practice may use a combination of on-site visits as well as remote video calls as described herein.

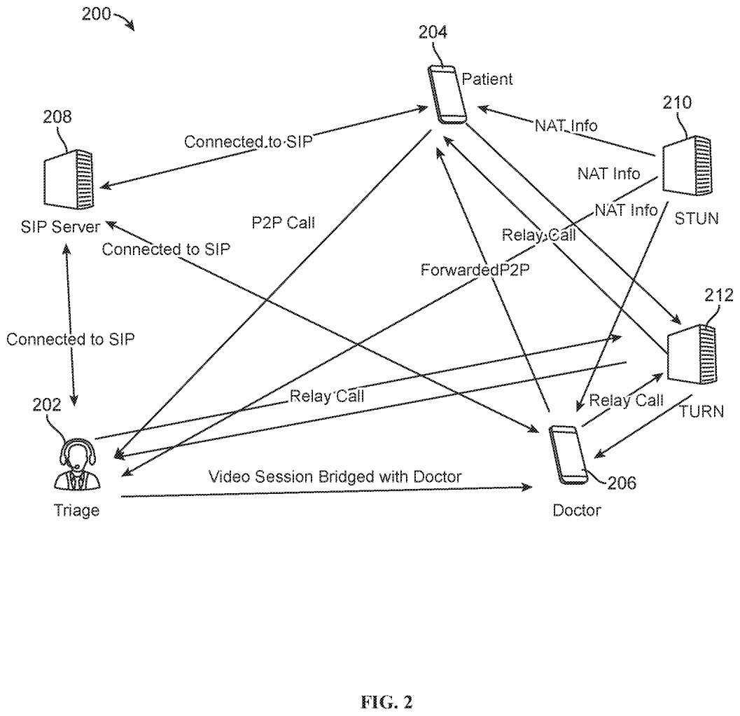

FIG. 2 is a diagram of a system and method in accordance with an embodiment of the present disclosure. FIG. 2 depicts components and interactions of a system 200, some components of which correspond with components of the system 100.

System 200 comprises a reception device 202, a drone device 204, a doctor device 206, a SIP server 208, a STUN 210, and a TURN 212. While the reception device 202, the drone device 204, and the doctor device 206 appear in FIG. 1 to be mobile devices, such depiction is provided for illustration and discussion purposes only and in embodiments these components are not mobile devices. The reception device 202 is labeled Triage in FIG. 2 and appears as a person wearing a headset as in a call center setting. Embodiments of the present disclosure include triage operators working in call center environments and using instances of the web console 118 to provide the services provided herein. The SIP connections, relay calls, and P2P calls between the components are readily visible in FIG. 2.

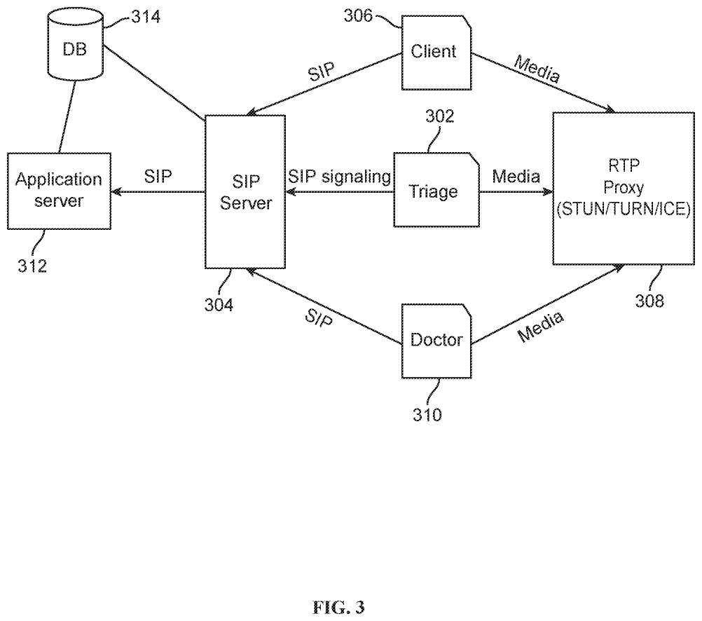

FIG. 3 is a diagram of a system in accordance with an embodiment of the present disclosure. FIG. 3 depicts components and interactions of a system 300, some components of which correspond with components of the system 100 and the system 200.

Components of the system 300 comprise reception 302, SIP server 304, client 306, RTP proxy (STUN/TURN/ICE) 308, doctor 310, application server 312, and database 314. The preceding discussion regarding components of the system 100 and the system 200 and their interactions applies to the components of the system 300 and their interactions.

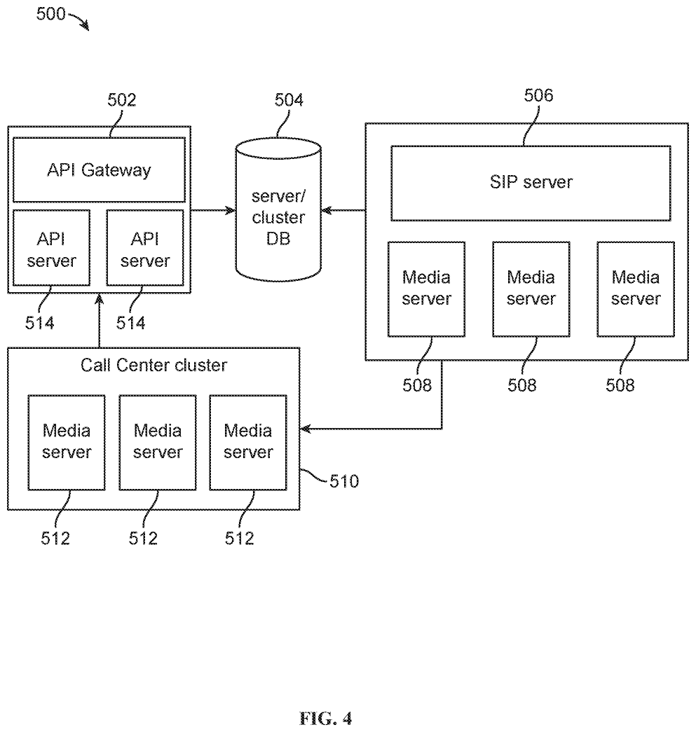

FIG. 4 is a diagram of a system and method in accordance with an embodiment of the present disclosure. FIG. 4 depicts components and interactions of a system 500 comprising an API Gateway 502, a server/cluster DB 504, a SIP server 506, and RTP proxies 508. The system 500 also comprises a call center cluster 510, media servers 512, and API servers 514. FIG. 4 depicts interactions between web console/doctor patient devices and call handing using the custom API server and backend server of real-time handling of events.

In embodiments that may be illustrated at least by FIG. 4, two video-over-IP or other sessions may be involved instead of one. Instead of the session originated by the drone device 106 being received by the server 102 and the server then conferencing the provider device 110 into the session, the drone device 106 in alternative embodiments originates a second session directly with the provider device 110 while the first session still ongoing. The two sessions are then merged, and all three parties can then communicate together. In this alternative embodiment, both the first and second video-over-IP sessions are technically originated by the drone device 106. The API 122 executing on the backend server 120 causes the drone device 106 to initiate the second session to contact the provider device 110. This process may not be visible or otherwise perceptible to the user of the drone device 106. The user may perceive that he/she is transitioned from a hold state with the triage operator to a video session with the provider and the provider device 110. In some embodiments, the first video session that the drone device 106 initiated with the triage server 102 terminates at about the time the second video session is established such that the triage operator may effectively drop out of the call and the patient and provider begin conversing.

In embodiments, two or more providers may collaborate in providing care to a patient. For example, pathologists may upload radiological images and blood tests to a case file for the primary specialist to view. These materials may be linked by QR code, by patient driver license number or by other means, such as a secret personal identification number (PIN) provided to the drone device 106.

In some embodiments, a portable health station may be placed in a rural community health center. Persons in need of services may travel from their farms or other distant locations to the community health center in their village. The portable health station may receive drone devices 106 and other equipment for use in communicating with distant providers. Some drone devices 106 may have integrated sensors or be connected to nearby sensors and devices to detect and report, for example, blood pressure, glucose levels and electrocardiogram data. For example, a drone device 106 may have a Bluetooth or other wireless network interface for pairing with such sensors and devices for retrieval of health data, and the drone device 106 may then report the health data to provider devices 110.

For rural patients, such a program may allow faster service with the confidence that the patients are dealing with legitimate healthcare providers. For doctors and other providers, they may provide service from any location at any time instead of from their offices only and limited to office hours.

A nation such as Indonesia, with a population of nearly 270 million, suffers from high rates of heart and cardiovascular disease. Infant mortality is also high. Because the population is spread across thousands of islands, many persons do not receive medical checkups including heart monitoring. Further, Indonesia has a very small number of cardiologists and most are located in large cities. As for newborn delivery, one resolution to this problem could be outfitting rural health clinics with fetal biometry detection equipment and conducting examinations before consulting with expert physicians in distant areas. The systems and methods provided herein may assist in delivering care in these situations.

While the system 100 and FIG. 1 provided only one instance of the web console 118, systems and methods provide for a plurality of web consoles 118. In embodiments, the triage operator as described herein may be one of many such operators that work in a call center or work-at-home environment as part of a team. The triage operator performs a recommend/match on a multitude of experts/doctors by accessing at least the database 114 and using search engines and other available electronic tools.

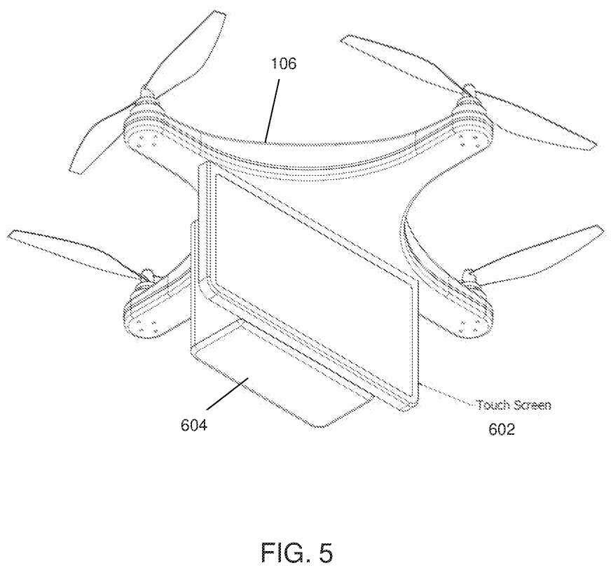

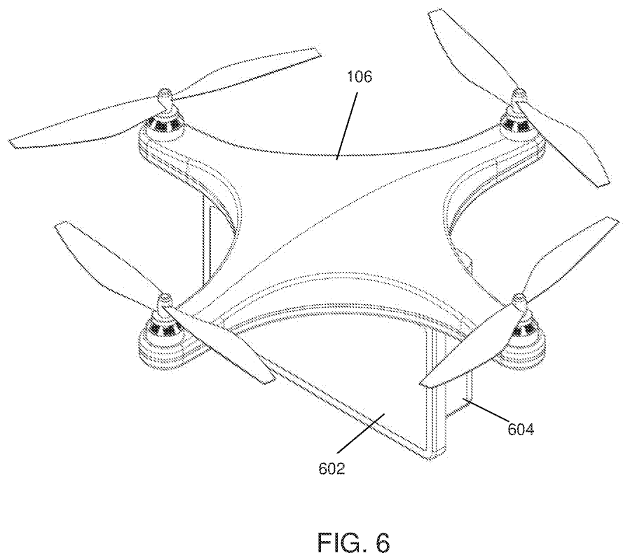

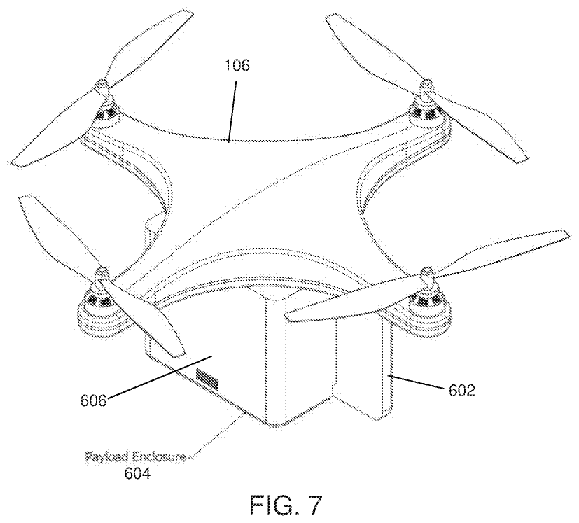

FIG. 5, FIG. 6 and FIG. 7 depict an example of a drone device 106 that may be used in many embodiments herein. Suspended from a lower surface of the drone device 106 are components that may be used in the execution of methods described herein. The communications component 108 may be included in such components which may include a touch screen 602 as indicated in the bottom perspective view of FIG. 5. The remotely located person who receives the drone device 106 may use the touch screen 602 to enter instructions, media, or communications to be transmitted to the provider device 110 or the triage server 102, for example.

The drone device 106 also comprises a payload enclosure 604 (as best illustrated in the rear perspective view of FIG. 7) for containing items to be dispensed to the remotely located person, for example medications, test kits, and/or paper instructions. The payload enclosure 604 may have a cover 606 coupled to an actuator (not shown) which is configured to receive control signals from a processor of the drone device 106, for example to unlock the cover 606 such that the contents can be retrieved.

The drone device 106 may implement (for example, in conjunction with the triage server 102 and/or a provider device 110) a security protocol to prevent unauthorised access to the contents of payload enclosure 604. For example, a one-time security code may be generated at the triage server 102 or the provider device 110, and communicated to the remotely located person. The communication of the security code may be via the drone 106 itself, with the remotely located person then required to enter the security code via touch screen 602 to unlock the cover 606 of payload enclosure 604. Alternatively, the security code may be communicated via an out-of-band channel, such as by SMS to the mobile phone of the first responder or family member who sent the SMS to the triage service center to initiate the dispatch of the drone 106.

FIG. 6 depicts the drone device 106 shown in FIG. 5 from a different angle (a front perspective view), while FIG. 7 depicts the drone device from a further different angle (a rear perspective view).

Many modifications will be apparent to those skilled in the art without departing from the scope of the present disclosure.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A system for video conferencing and real-time session bridging for services delivery, comprising:
at least one processor;
a memory; and
an application stored in the memory that when executed on the at least one processor:
dispatches, based on receiving a request for assistance at a remote location, an unmanned aerial vehicle to the remote location, the unmanned aerial vehicle containing two-way communication functionality;
joins a communication session initiated by the unmanned aerial vehicle;
determines, based on information exchanged during the communication session, a provider device to assist a patient;
enabling the provider device to be joined to the communication session; and,
enabling the unmanned aerial vehicle to dispense materials to the patient at the instruction of at least one of: a user of the provider device or a triage function.

2. The system of claim 1, wherein the unmanned aerial vehicle is a drone device programmed to depart the remote location when the communication session ends.

3. The system of claim 1, wherein the application receives at least one of voice, video, and text media for the user of the provider device to render assistance to the patient during the communication session.

4. The system of claim 1, wherein the processor is associated with the triage function that is configured to make determinations of the provider device to engage with the patient.

5. The system of claim 4, wherein the triage function is configured to search databases to evaluate provider devices as candidates to engage with the patient.

6. The system of claim 3, wherein the user of the provider device is a healthcare provider.

7. A method for video conferencing and real-time session bridging for services delivery, comprising:
a computer joining a wireless communication session initiated by a remotely located device;
the computer, based on information provided by the device, determining a first provider device to engage the remotely located device;
the computer causing the first provider device to be joined to the communication session; and
the computer, at least upon advice issued by the first provider device, causing materials to be dispensed by the remotely located device to a patient.

8. The method of claim 7, wherein the remotely located device is an unmanned aerial vehicle.

9. The method of claim 7, wherein the remotely located device is dispatched by the computer to the patient.

10. The method of claim 7, wherein the information provided by the patient describes a problematic situation experienced or to be experienced by the patient.

11. The method of claim 7, wherein a user of the first provider device is knowledgeable in the situation of the patient.

12. The method of claim 7, wherein the computer reviews information about a plurality of provider device users prior to causing the first provider device to be joined to the communication session.

13. A method for video conferencing and real-time session bridging for services delivery, comprising:
a provider device causing an unmanned aerial vehicle to be sent to a remote user;
the provider device, after the unmanned aerial vehicle is received by the remote user, joining a communication session initiated by the remote user to a triage server;
the provider device engaging in direct communication with the remote user; and
the provider device, based at least on the direct communication, causing the unmanned aerial vehicle to dispense materials to the remote user.

14. The method of claim 13, wherein the unmanned aerial vehicle is a drone device with two-way communication functionality.

15. The method of claim 13, wherein triage server, upon joining the session initiated by the remote user, joins the provider device to the session.

16. The method of claim 13, wherein the direct communication engaged in by the provider device with the remote user comprises a user of the provider device diagnosing a problem reported by the remote user.

17. The method of claim 13, wherein the triage server previously selected the provider device from a plurality of provider devices to provide assistance to the remote user.

18. The method of claim 13, wherein the communication session further comprises the remote user, the provider device, and the triage server exchanging at least one of still images and video content.

19. The method of claim 13, wherein the unmanned aerial vehicle returns to the triage server when the communications session ends.

20. Non-transitory computer readable storage having stored thereon instructions for causing at least one processor to perform the method according to claims 13.
